(19) United States
(12) Patent Application Publication
(10) Pub. No.: US 2020/0046675 A1
(43) Pub. Date: Feb. 13, 2020

(54) Cannabinoid Formulation Packaged in a Glass Vial For Pulmonary Delivery

(71) Applicant: WaxNax LLC, Denver, CO (US)

(72) Inventors: Alex Kaplan, Lakewood, CO (US); Turner Miller, Superior, CO (US); Shane Vogt, Lakewood, CO (US)

(21) Appl. No.: 16/102,286

(22) Filed: Aug. 13, 2018

(51) Int. Cl. A61K 31/352; A61K 47/44; A61K 9/00

(52) U.S. Cl. CPC A61K 31/352; A61K 9/0073; A61K 47/44

(57) ABSTRACT

A cannabinoid formulation comprising cannabinoid concentrate and beeswax, or a beeswax analogue, in a ratio of 30:1 to 40:1 by weight. Preferably the ratio is 35:1. The vaporizable formulation is packaged in a single use glass vial and used for pulmonary administration to a patient. The glass vial is made from borosilicate glass, has a cylindrical shape, an open end and no lid. The vial preferably has a uniform wall thickness of 0.02 inches. The formulation forms a wax seal at room temperature to eliminate any need for a lid. The formulation can be directly vaporized from the glass vial to eliminate transfer of the formulation to another container, to eliminate mess, to enable precise dosing, and to enable easy recycling and disposal of the vial after use.

### FIELD OF THE INVENTION

The present invention is generally directed to vaporizable formulation including cannabinoid concentrates and beeswax, and packaging for use in a vaporization device.

### BACKGROUND OF THE INVENTION

Cannabis, commonly known as marijuana, hemp, and by numerous other names, is used as a drug and as medicine. The constituent of Cannabis is tetrahydrocannabinol (THC), including many other cannabinoids, such as cannabidiol (CBD), cannabinol (CBN), tetrahydrocannabivarin (THCV) and cannabigerol (CBG).

The medicinal and psychoactive properties of the Cannabis have been known for centuries. Further, the cannabinoids are known for treating disease, disorder or certain medical conditions. The illegality of Cannabis has prevented wide spread usage. At present, Cannabis is not legally available in most countries because many Cannabis-based products contain harmful substances, stabilizers, artificial flavorings, colors, toxins, as well as toxic byproducts from extraction methods, and have side effects and measurably failed to provide effective for treating disease, disorder or certain medical conditions.

However, there is growing legalization of its use, especially for medicinal purposes. Therefore, it is to legalize Cannabis for medical use, there is a need to develop Cannabis for the effective with improved efficacy and non-toxic for human consumption.

Various types of compositions of cannabinoids are known in the prior art for use in therapeutic treatment of disease, disorder or various medical conditions, such as, for example, as an analgesic for treatment of arthralgia, neuralgia, inflammation, for inducing appetite, treatment of sleep apnea, hypertension, inhibiting growth of cancerous cells, anorexia, AIDS, chronic pain, muscle spasticity, glaucoma, arthritis, migraine and many other medical treatments and therapies.

The pharmacological and therapeutic properties of cannabinoids are undergoing substantial discovery over the last few decades and are subject to a growing amount of scientific research. There remains an unmet need for a measurable, reproducible and sustained release vapors form of vaporizable cannabinoid for the treatment of disease, disorder or medical conditions.

Often, Cannabis patients require rapid dosing of cannabinoids to achieve the desired therapeutic effects. In cases of nausea and/or acute pain, rapid cannabinoid delivery is essential. In these cases, patients will generally inhale vaporizable Cannabis and maximum blood plasma levels can be achieved in minutes.

Therefore, there is a need for vaporizable formulation of cannabinoids which is efficient and stable can affect the rate and efficacy of which vaporizes the product for pulmonary administration.

### BRIEF DESCRIPTION OF THE DRAWINGS

FIG. 1 is a top perspective view of a glass vial in accordance with the present invention.

FIG. 2 is side view of the glass file of FIG. 1 filled with a predetermined quantity of the formulation of the present invention.

FIG. 3 is side view of the glass file of FIG. 1 filled with a predetermined quantity of the formulation of the present invention and having a wax seal at room temperature.

### SUMMARY OF THE INVENTION

The present invention includes formulations and compositions comprising Cannabis concentrates and beeswax. In some embodiments, the composition or formulation is a pharmaceutical composition or pharmaceutical formulation. In some embodiments, the compositions described herein are provided for pulmonary administering to a patient. In some embodiment, the composition is vaporizable formulation prepared by mixing cannabinoid concentrate with beeswax.

In one aspect of the present invention a vaporizable cannabinoid formulation is prepared by mixing cannabinoid concentrate with beeswax where the ratio of cannabinoid concentrates and beeswax is at least 5:1 by weight.

Further, in one aspect of the present invention the ratio of cannabinoid concentrates and beeswax is 80:1 by weight.

Further, in one aspect of the present invention the ideal ratio of cannabinoid concentrates and beeswax is 35:1 by weight percentage.

In another aspect of the present invention is a method for preparing the vaporizable formulation by mixing the cannabinoid concentrate with the beeswax in a container or any other similar article and make sure that the cannabinoid concentrate is properly mixed with the beeswax.

The mixed composition is then transfer into an automatic filling machine. The machine is used for precisely filling the composition into glass vials. Once the glass vials are filled with predetermined quantity, the composition is then cooled until hardened. After that the glass vials are packaged into the custom joint tube.

In another aspect, the beeswax is mixed with one or more of the following, raw oil, organic oil, shatter, wax, essential oils and other extracts of Cannabis sativa l, or other plant material. The base elements act as emulsifier. When the formulation cools from a heated condition, the beeswax, having a lesser density, will float to the top and create a seal atop the formulation in a glass vial packaging. This automatically seals the product for use in a dabbing device, or other vaporization device, which vaporizes the product for pulmonary administration. The prepared vaporizable formulation is then used for pulmonary administration to a patient or user.

In another aspect of the invention, the formulation is packaged in at least one customised glass vial, the glass vial has a minimum wall thickness of 0.02 inches to inhibit catastrophic thermal stress during use. Preferably the glass vials have 0.022 inches in wall thickness, 0.315 inches in diameter and 0.413 inches in length. These dimensions, combined with a low coefficient of thermal expansion makes the glass vial usable not only as a package, but the formulation can be vaporized directly from the glass vial without the glass vial cracking, breaking, melting, or otherwise experiencing catastrophic thermal stress.

In another aspect, beeswax is mixed with the cannabinoid concentrate as an excipient, and an aliquot of the excipient that will rise, harden and create a seal during packaging of the formulation. The mixing of beeswax with the cannabinoid concentrate is to improve the efficacy and non-toxic when vaporized or burned. The use of beeswax, or other wax, eliminates the need for a closure or lid on the glass vial. This further enables the use of the glass vial as packaging and functioning housing during vaporizing the formulation.

It will be understood that certain ingredients can be added to the compositions described herein without materially affecting the basic and novel properties of the compositions described herein. For example, the compositions can include undisclosed and/or unclaimed ingredients that do not materially affect the basic and novel properties of the compositions described herein, therapeutic or otherwise. Examples of such ingredients include flavorings and sweeteners that provide a more pleasant taste and/or odor, but do not materially affecting the desired properties, therapeutic or otherwise, of the compositions described herein.

Other variations, embodiments and features of the present disclosure will become evident from the following detailed description, abstract and claims.

### DETAILED DESCRIPTION

The term “formulation” includes the compositions described herein and any additional components that are desired for administering the composition to a user or for consumption of the composition by a user.

The term “treating” or “treatments” as used herein can include any of the following: alleviating, reducing, improving, mitigating, or eliminating a disease, disorder or medical condition.

The term “cannabinoid concentrate” or “cannabinoid extract” as used herein can include any concentrated form of Cannabis or Cannabis derivatives. In various embodiments, the cannabinoid concentrate has at least 30% bio active cannabinoids by weight, and preferably at least 60% bio active cannabinoids by weight.

The Cannabis extract comprises tetrahydrocannabinol (THC), and/or other cannabinoids, such as cannabidiol (CBD), cannabinol (CBN), tetrahydrocannabivarin (THCV) and cannabigerol (CBG). In some embodiments, the extract comprises natural, neutral, or acidic forms of the cannabinoids, or semi-synthetic and synthetic derivatives thereof. In some embodiments, the Cannabis extract comprises terpenes and/or flavonoids.

In some embodiments, the formulation described herein includes a whole plant cannabinoid extract. In other embodiments, the cannabinoid extract is a THC or CBD isolate fortified with particular additional cannabinoids and terpenes.

The formulations described herein are useful for the treatment and prevention of a wide range of diseases, disorders or medical conditions, including, for example, inflammation, inflammatory bowel disease (IBS), Crohn's disease (CD), irritable bowel syndrome (IBS), ulcerative colitis (UC), nausea, vomiting, anorexia, cachexia, treating pain such as cancer pain, bone pain, neuropathic pain, migraine, headaches, postmenstrual syndrome (PMS), Cancer, neurodegenerative diseases like Lou Gehrig's disease, Huntington's disease, Alzheimer's dementia, Parkinson's disease and Parkinsonian-type symptoms, spinal-cord injuries; HIV/AIDS, agitation, insomnia, depression, muscle spasms, spasticity from multiple sclerosis, glaucoma, Autism Spectrum Disorder (ASD), Attention Deficit Hyperactivity Disorder (ADHD), Post-Traumatic Stress Disorder (PTSD), and anxiety disorders. Those skilled in the art will recognize that the embodiments described herein may be used to treat many diseases, disorders or medical conditions that respond favorably thereto.

The cannabinoid and/or terpene concentrates obtained from extraction from Cannabis and formulation of the concentrates, particularly for use for direct vaporization, infusion into edible matrices, in electronic inhalation devices, and as nutraceuticals. Additionally, the process for preparing cannabinoid and/or terpenoid extracts for human consumption includes, concentrating glandular trichomes from Cannabis plant material; passing a solvent though the plant material to produce a cannabinoid/terpenoid-containing eluate, filtering and refining the eluate to remove undesirable impurities and processing the eluate into a consumable form.

The embodiments described herein demonstrate an improved efficacy that is unexpected compared to utilizing the same dose of the existing cannabinoid and/or terpene concentrates.

In one embodiment, the present invention provides a vaporizable formulation prepared by mixing cannabinoid concentrate with beeswax.

Beeswax is an amazing natural substance that can be used in many ways to improve our lives. Beeswax is a natural wax produced in the bee hive of honey bees. Beeswax is mainly esters of fatty acids and various long-chain alcohols. Its main components are palmitate, palmitoleate, and oleate esters of long-chain (30-32 carbons) aliphatic alcohols. Particularly, the ratio of triacontanylpalmitateCH₃(CH₂)₂₉O—CO—(CH₂)₁₄CH₃ to cerotic acid CH₃(CH₂)₂₄COOH, the two principal components, being 6:1 in one embodiment of the invention. In an alternate embodiment, the ratio is between 5:1-7:1. Purified and bleached beeswax is used in the production of food, cosmetics, and pharmaceuticals and is a preferred excipient in the present formulation. It can be appreciated than any salt or ester of palmitic acid can be substituted for the triacontanylpalmitate in accordance with the present invention.

In an alternate embodiment of the invention, a beeswax analogue is utilized including palimitate, palmitoleate, and oleate esters of long-chain aliphatic alcohols. The approximate ratio of triacontanylpalmitate to cerotic acid is 6:1. In an alternate embodiment, the ratio is between 5:1-7:1.

Beeswax is considered safe for consumption and has been approved as an ingredient in human food in the USA and many countries. It is generally inert, i.e. it does not interact with the human digestive system at all and passes through the body unaltered. If mixed with medicinal drugs or poisonous baits, wax preserves the active materials longer and releases them slowly. It allows the mixture of very stable and smooth emulsions for cosmetics and medical drugs. It has anti-inflammatory and antioxidant activity and beeswax is non-toxic when vaporized or burned.

The beeswax has interesting electro-chemical properties. The charge of ions produced when vaporized may have beneficial bioactivity. As person skilled in the art already knows that beeswax is the all-natural, non-toxic waxy substance secreted by bees. Beeswax produces negative ions that help freshen and purify the air. Further, negative Ionization helps eliminate dust, mold and bad odor floating in the atmosphere, easing allergy and asthma symptoms and improving breathing for anyone around. They do not produce any toxic byproducts and heavy soot when burned.

In some embodiments, the present invention provides a vaporizable formulation prepared by mixing cannabinoid concentrate with beeswax. In some embodiment, the ratio of cannabinoid concentrates and beeswax is between 5:1 to 80:1 by weight. In one embodiment, the ratio of cannabinoid concentrates and beeswax is 30:1 by weight percentage. More preferably, the ratio of cannabinoid concentrates and beeswax is 35:1.

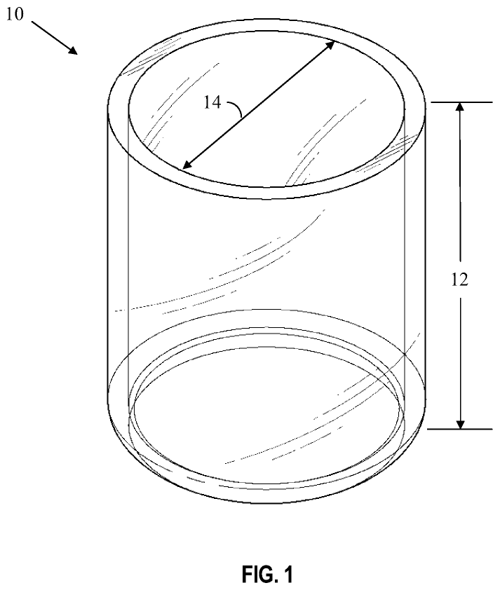

FIG. 1 shows the vial 10 having a diameter 14 and a length 12. The borosilicate glass is transparent, or semi-transparent. In one embodiment, the shape is cylindrical for structural integrity and ease with uniform heating of the contents with a heated rod or needle inserted axially into the vial. The precise shape and relative dimensions of 0.022 wall thickness, a 0.315 diameter 14 and a 0.413 length 12 with all dimensions in inches in one variant of the invention. With a 0.315 inch diameter, a heated rod can readily vaporize the contents while maintaining a consistent distance from all walls of the vial. In addition to delivering uniform heat to the contents, the vial is uniformly heated along all walls to inhibit cracking or other structural failure. It can be appreciated that the proportions of the present invention can be modified to accommodate dosage and vaporization temperature needs of a subject, patient, or user.

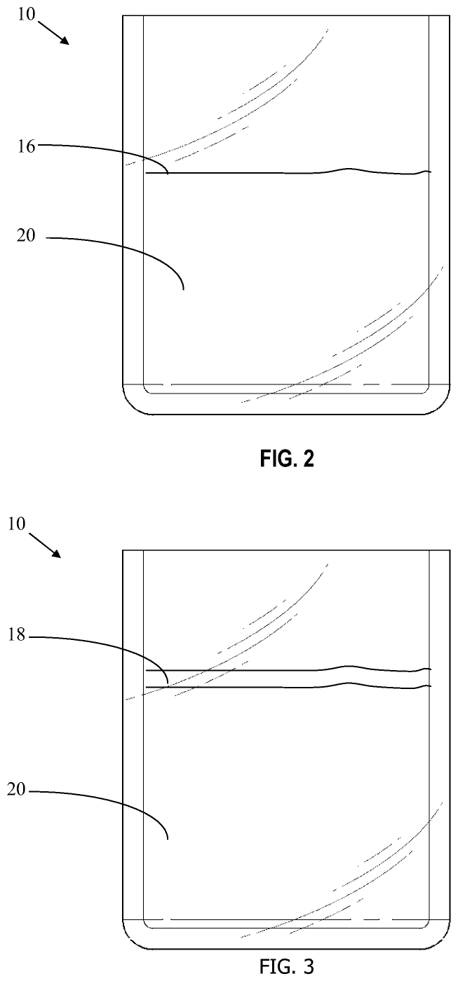

FIG. 2 shows the vial 10 filled with a vaporizable formulation 20. The formulation 20 has a surface 16. The formulation 20 is a combination of Cannabis extract and beeswax, or analogue thereof. It is in a uniform concentration distribution initially when manufactured at above-ambient temperatures such as between 140-150 degrees F.

FIG. 3 show the vial 10 after filling. The vaporizable formulation 12 cools. During the cooling process a portion of the beeswax floats to the surface to create a beeswax seal 18 having a thickness capable of maintaining an airtight seal between the formulation 20 and the exterior of the vial 10. Maintaining an airtight seal inhibits contaminants from entering the formulation 20 and also inhibits the volitization of terpenes, flavinoids and other components of the formulation and enhances shelf life. The beeswax seal also inhibits oxidation of the cannabinoids and various components of the formulation 20.

In some embodiments, the cannabinoid concentrate is mixed with the beeswax in a container or any other similar article and make sure that the cannabinoid concentrate is properly mixed with the beeswax. The mixed composition is then transfer into an automatic filling machine. The machine is used for precisely filling the composition into glass vials. Once the glass vials are filled with predetermined quantity, the composition is then cooled until hardened. After that the glass vials are packaged into the custom joint tube.

In some embodiments, the automatic filling machine delivers a programmed amount of composition and enables controlled dosing in multi-generational product lines. This technology gives excellent quality control and reproducibility, hallmarks of pharmaceutical standards.

In some embodiments the glass vials have a wall thickness of at least 0.02 inches. Preferably the glass vials are cylindrical in shape with a closed end, a 0.022 inch wall thickness, a diameter of 0.315 inches and a length of 0.413 inches.

Benefits of glass vial packaging of the present invention include precise dosing. The glass vials are sized to house a precise dose of cannabinoid formulation, and are readily disposable or recyclable as glass.

The elimination of any need for an additional closure or lid eliminates non-recyclable waste, and reduces packaging cost. Further the use of the disposable glass vial eliminates mess normally associated with dosing Cannabis concentrate, which can otherwise be sticky, oily and cumbersome to handle. Using the glass vial for packaging and consumption eliminates the need to transfer the product between separate containers for use and keeps any vaporization device clean and residue free.

The lid-free vial design saves manufacturing and packing cost, which can be passed to the consumer. During operation (vaporizing) the formulation, the open vial cylindrical design allows heat to readily and uniformly penetrate the formulation. This vial design also allows optimization of pulmonary administration. In one embodiment, the glass vials have a minimum coefficient of thermal expansion of at least 2×10⁻⁶ K⁻¹ at 20° C. to resist thermal shock when the vial is used in a vaporization device to vaporize the formulation contained therein. In another embodiment, the glass vials have a coefficient of thermal expansion within the range of between 2×10⁻⁶ K⁻¹ thru 5×10⁻⁶ K⁻¹ at 20° C. to resist thermal shock. In one embodiment the glass is fabricated from borosilicate glass having a B₂O₂ concentration of 8-25%, and a SiO₂ concentration of between 65-75%.

Preferably the vial glass has a melting point at above 1000° F. The wall thickness is between 0.10-0.60 inches because thickness of less than 0.010″ are expensive and difficult to manufacture. The thickness of less than 0.10 lacks structural integrity. Glass thicker than 0.060 inches provides inadequate heat transfer for optimal vaporizing of the contents.

In one embodiment, the glass vial is manufactured from optical quartz glass, or fused silica glass. This type of glass has a melting point of approximately 1585° C., and can operate safely at temperatures of less than 900° C. Optical quartz glass is durable, has a low electrical conductivity, and is transparent. Preferably, the glass thickness is at least 0.02 inches thick, and more preferably between 0.01-0.02 inches.

In some embodiments, one or more glass vials are packaged into a joint tube. However, in some embodiments, one joint tube is configured to include seven glass vials.

In some embodiments, an aliquot of beeswax is mixed with the cannabinoid concentrate, and an aliquot that will rise, harden and create a seal. The mixing of beeswax with the cannabinoid concentrate is to improve the efficacy and non-toxic when vaporized or burned.

In some embodiments, the beeswax is further mixed with one or more of the following, raw oil, organic oil, shatter and wax. These additional elements enable emulsification of the formulation. When the formulation cools from a heated condition, the beeswax floats to the top of the formulation in the vial to create a vaporizable seal in the vial. This automatically seals the product for prolonged storage and can also be vaporized along with the formulation in a dabbing device, or other vaporizing device for pulmonary administration.

The composition is inhaled by means of inhaler or any other similar device, enabling delivery of composition via the airways into the bloodstream. Increasingly, the composition is for systemic delivery, taking advantage of the optimal permeability into blood stream. Further, the large surface area of the lung, combined with the extensive blood flow and extremely thin alveolar-capillary membrane, means that composition in particular are absorbed within seconds, making inhaled delivery particularly suitable for conditions in which the patient would benefit from a rapid response.

In some embodiments, the formulation is vaporized for pulmonary administration in a user or a patient. In some embodiments, the melting point of the excipient is within the range of 125-160° F., or preferably within the range of 140-150° F. and the vaporization point of the excipient e.g. beeswax is within the range of 400-800° F., or preferably within the range of 400-600° F.

Alternatives to beeswax in accordance with the present invention include beeswax analogues synthesized or derived from natural sources. These can be mixed with, or formulated from, waxes derived from Cannabis sativa L to form the excipient. In one embodiment the beeswax analogue is manufactured from elements of the group consisting of: naturally derived plant waxes, coconut oil, palm oil, palm kernel oil, cocoa butter, lard, gelatin, polyethylene glycol, propylene glycol, glycerol, ethylene glycol and other excipients such as those used in e-cigarettes, and combinations thereof.

Preferably the formulation including the excipient has a viscosity above 10,000 cP (centipoises) at 80° F. Honey has a viscosity on the order of 10,000 cP at 80° F. for comparison purposes.

In an alternate embodiment, the formulation is solid at below 110° F., having a vaporization temperature within the range of between 450-1,000° F. In this embodiment, any suitable combination of the above listed excipients can be used.

The embodiments described above demonstrate an improved efficacy that is unexpected compared to utilizing the same dose of the same active source of cannabinoid concentrate.

In embodiments, the composition comprises a pharmaceutical product; the dosage forms described herein provide clear separation from the confusion associated with traditional preparations of natural cannabinoid infused products.

Any alterations and further modifications of the compositions and/or formulations described herein, which would normally occur to one skilled in the relevant art and having possession of this disclosure, are to be considered within the scope of the instant claims.

While particular elements, embodiments and applications of the present invention have been shown and described, it will be understood, of course, that the invention is not limited thereto since modifications can be made by those skilled in the art without departing from the scope of the present disclosure, particularly in light of the foregoing teachings. In particular beeswax can be replaced with a beeswax analogue being naturally or synthetically derived. Further, the present invention can be used for non-Cannabis formulations capable of being vaporized for pulmonary delivery.

1. A packaged cannabinoid formulation comprising a composition of cannabinoid concentrate and beeswax, wherein the ratio of the cannabinoid concentrate and the beeswax is in a range of 5:1 to 80:1;
the formulation is packaged in a cylindrical glass vial having a beeswax seal to enable the formulation to be vaporized directly from the cylindrical glass vial; and
the glass vial has a coefficient of thermal expansion within the range of between 2×10⁻⁶ K⁻¹ thru 5×10⁻⁶ K⁻¹ at 20° C. to resist thermal shock and to enable the formulation to be vaporized directly from the cylindrical glass vial.

2. The formulation of claim 1, wherein the cannabinoid concentrate and the beeswax have a ratio of, at least, 5:1 by weight.

3. The formulation of claim 1, wherein the cannabinoid concentrate and the beeswax have a ratio of, at most, 80:1 by weight.

4. The formulation of claim 1, wherein the cannabinoid concentrate and the beeswax have a ratio of between 30:1 to 40:1 by weight.

5. The formulation of claim 1, wherein a melting point of the beeswax is in range of 125-160° F.

6. The formulation of claim 1, wherein a vaporization point of the beeswax is in range of 400-800° F.

7. The formulation of claim 1, wherein the composition is vaporized for pulmonary administration in a patient.

8. A method of treating a disease, disorder or medical condition, the method comprising pulmonary administration of a therapeutically effective amount of the formulation of claim 1 to a patient.

9. A vaporizable formulation comprising a composition of cannabinoid concentrate and a beeswax analogue, wherein the ratio of the cannabinoid concentrate and the beeswax analogue is in a range of 5:1 to 80:1; and
the beeswax analogue includes triacontanylpalmitate and cerotic acid, with the ratio of triacontanylpalmitate CH₃(CH₂)₂₉O—CO—(CH₂)₁₄CH₃ to cerotic acid CH₃(CH₂)₂₄COOH being between 5:1 to 7:1, and
the formulation is packaged in a cylindrical glass vial having a wax seal to enable the formulation to be vaporized directly from the cylindrical glass vial.

10. The formulation of claim 9, wherein the cannabinoid concentrate and the beeswax analogue have a ratio of 5:1 by weight.

11. The formulation of claim 9, wherein the cannabinoid concentrate and the beeswax analogue has a ratio of 80:1 by weight.

12. The formulation of claim 9, wherein the cannabinoid concentrate and the beeswax analogue has a ratio of 35:1 by weight, and the ratio of triacontanylpalmitate CH₃(CH₂)₂₉O—CO—(CH2)14CH3 to cerotic acid CH₃(CH₂)₂₄COOH is approximately 6:1.

13. The formulation of claim 9, wherein a melting point of the beeswax analogue is in range of 125-160° F.

14. The formulation of claim 9, wherein a vaporization point of the beeswax analogue is in range of 400-800° F.

15. A method of treating a disease, disorder or medical condition, the method comprising pulmonary administering a therapeutically effective amount of the formulation of claim 9 to a patient.

16. The formulation of claim 9, wherein the glass vial is manufactured from borosilicate glass having a wall thickness of at least 0.01 inches.

17. The formulation of claim 9, wherein the glass vial has a coefficient of thermal expansion within the range of between 2×10-⁶K-¹ thru 5×10-⁶K-¹ at 20° C. to resist thermal shock.

18. The formulation of claim 17, wherein the glass vial has a 0.022 inches in wall thickness, 0.315 inch diameter, and 0.413 inch length to optimize heat transfer during vaporization of the formulation in the glass vial.

19. The formulation of claim 9, wherein the glass vial is manufactured from quartz glass having a wall thickness of between 0.01 to 0.02 inches.

20. The formulation of claim 9, wherein the glass vial is manufactured from quartz glass having a wall thickness of at least 0.02 inches.
